(19) United States
(12) Patent Application Publication
(10) Pub. No.: US 2022/0067866 A1
(43) Pub. Date: Mar. 3, 2022

(54) GROUP SYNCHRONIZED DISTRIBUTION OF PRESCRIPTION MEDICINES

(71) Applicant: Philip J. Baker, Millington, TN (US)

(72) Inventor: Philip J. Baker, Millington, TN (US)

(21) Appl. No.: 17/006,251

(22) Filed: Aug. 28, 2020

(51) Int. Cl. G06Q 50/28; G16H 20/10; G06Q 10/10; G06Q 30/02; G06Q 20/08

(52) U.S. Cl. CPC G06Q 50/28; G16H 20/10; G06Q 20/085; G06Q 30/0206; G06Q 10/1057

(57) ABSTRACT

Various methods for supplying prescription medicines to members of a group in a synchronized manner are described. A method for supplying prescription medicines to members of a group in a synchronized manner includes defining a supply interval, defining one or more fixed delivery dates for a given year based on the supply interval, defining a regular prescription supply number of days, defining a set of member prescriptions for each member of the group to create a plurality of member prescriptions, separating a regular prescription supply of the relevant prescription medicine from a bulk supply of the prescribed medicine for each member prescription of the plurality of member prescriptions to create a plurality of regular prescription supplies, placing each regular prescription supply of the plurality of regular prescription supplies into a container, and sending each container to the member of the group having the prescription for the regular prescription supply contained within the container for delivery on the next occurring fixed delivery date of the one or more fixed delivery dates. Prescription medicine distribution systems are also described.

### FIELD

The disclosure relates to the field of prescription medicine distribution. More particularly, the disclosure relates to methods for supplying prescription medicines to consumers that are members of a defined group, such as employees of a common employer. Specific examples relate to methods for supplying prescription medicines to members of a group in a synchronized manner. Prescription medicine distribution systems are also described.

### BACKGROUND

Distribution of prescription medicines is currently undergoing significant change as pharmacies, which are responsible for the final delivery of medicines to the consumer, adopt new practices designed primarily to benefit consumers. For example, several methods and systems for aligning refill dates of multiple prescriptions for a consumer are known. Incorporation of these methods into the operations of retail pharmacies can provide several benefits to the consumer, including better health outcomes and a more convenient process for having prescriptions filled.

While adoption of these new practices has benefitted consumers of prescription medicine, it has created new operational challenges for retail pharmacies as consumers have fewer in-store visits. Furthermore, the prescription medicine supply chain has grown over the last several decades to include additional types of organizations, each adding to the overall cost of prescription medicines and contributing to the current negative impression many people have of the prescription medicine industry as a whole.

There is currently no existing method for supplying prescription medicines to consumers that achieves the desirable increase in benefits for consumers while also addressing the challenges that pursuit of these benefits has created for pharmacies.

A need exists, therefore, for improved methods for supplying prescription medicines and improved prescription medicine distribution systems.

### BRIEF SUMMARY OF SELECTED EXAMPLES

Various example methods for supplying prescription medicines are described.

An example method is a method for supplying prescription medicines to members of a group in a synchronized manner. This example method comprises defining a supply interval, defining one or more fixed delivery dates for a given year based on the supply interval, defining a regular prescription supply number of days, defining a set of member prescriptions for each member of the group to create a plurality of member prescriptions, separating a regular prescription supply of the relevant prescription medicine from a bulk supply of the prescribed medicine for each member prescription of the plurality of member prescriptions to create a plurality of regular prescription supplies, placing each regular prescription supply of the plurality of regular prescription supplies into a container, and sending each container to the member of the group having the prescription for the regular prescription supply contained within the container for delivery on the next occurring fixed delivery date of the one or more fixed delivery dates.

Another example method is a method for supplying prescription medicines to members of a group in a synchronized manner. This example method comprises charging a participation fee to a common prescription payer for the group, defining a supply interval, defining one or more fixed delivery dates for a given year based on the supply interval, defining a regular prescription supply number of days, defining a set of member prescriptions for each member of the group to create a plurality of member prescriptions, separating a regular prescription supply of the relevant prescription medicine from a bulk supply of the prescribed medicine for each member prescription of the plurality of member prescriptions to create a plurality of regular prescription supplies, placing each regular prescription supply of the plurality of regular prescription supplies into a container, and sending each container to the member of the group having the prescription for the regular prescription supply contained within the container for delivery on the next occurring fixed delivery date of the one or more fixed delivery dates.

Another example method is a method for supplying prescription medicines to members of a group in a synchronized manner. This example method comprises charging a participation fee to a common prescription payer for the group, defining a supply interval, defining one or more fixed delivery dates for a given year based on the supply interval, defining a regular prescription supply number of days, defining a set of member prescriptions for each member of the group to create a plurality of member prescriptions, charging a prescription fee for each member prescription of the plurality of member prescriptions to the common prescription payer for the group, separating a regular prescription supply of the relevant prescription medicine from a bulk supply of the prescribed medicine for each member prescription of the plurality of member prescriptions to create a plurality of regular prescription supplies, placing each regular prescription supply of the plurality of regular prescription supplies into a container, and sending each container to the member of the group having the prescription for the regular prescription supply contained within the container for delivery on the next occurring fixed delivery date of the one or more fixed delivery dates.

An example method is a method for supplying prescription medicines to members of a group in a synchronized manner. This example method comprises defining a supply interval, defining one or more fixed delivery dates for a given year based on the supply interval, defining a regular prescription supply number of days, defining a set of member prescriptions for each member of the group to create a plurality of member prescriptions, separating a regular prescription supply of the relevant prescription medicine from a bulk supply of the prescribed medicine for each member prescription of the plurality of member prescriptions to create a plurality of regular prescription supplies, placing each regular prescription supply of the plurality of regular prescription supplies into a container, for each member of the group, combining all containers containing a regular prescription supply for the member into a master container for the member, and sending each master container to the member for delivery on the next occurring fixed delivery date of the one or more fixed delivery dates.

Another example method is a method for supplying prescription medicines to members of a group in a synchronized manner. This example method comprises defining a subgroup of members from all members of the group for participation in the method, defining a supply interval, defining one or more fixed delivery dates for a given year based on the supply interval, defining a regular prescription supply number of days, defining a set of member prescriptions for each member in a defined subgroup of members of the group to create a plurality of member prescriptions, separating a regular prescription supply of the relevant prescription medicine from a bulk supply of the prescribed medicine for each member prescription of the plurality of member prescriptions to create a plurality of regular prescription supplies, placing each regular prescription supply of the plurality of regular prescription supplies into a container, and sending each container to the member of the group having the prescription for the regular prescription supply contained within the container for delivery on the next occurring fixed delivery date of the one or more fixed delivery dates.

An example method is a method for supplying prescription medicines to members of a group in a synchronized manner. This example method comprises defining a supply interval, defining one or more fixed delivery dates for a given year based on the supply interval, defining a regular prescription supply number of days, defining a set of member prescriptions for each member of the group to create a plurality of member prescriptions, for each prescription medicine represented in the plurality of member prescriptions, defining a total prescription supply, for each prescription medicine represented in the plurality of member prescriptions, acquiring the total prescription supply from a supplier of the prescription medicine, such as a manufacturer or a distributor, separating a regular prescription supply of the relevant prescription medicine from a the total prescription supply of the prescribed medicine for each member prescription of the plurality of member prescriptions to create a plurality of regular prescription supplies, placing each regular prescription supply of the plurality of regular prescription supplies into a container, and sending each container to the member of the group having the prescription for the regular prescription supply contained within the container for delivery on the next occurring fixed delivery date of the one or more fixed delivery dates.

Various prescription medicine distribution systems are also described.

An example prescription medicine distribution system comprises a computer having a processor, a computer readable memory, a database server and/or client application adapted to access a database server application, and computer program instructions that, when executed by the processor, retrieve data from the database server application, either directly or via the database client application, relating to a supply interval, one or more fixed delivery dates, one or more member prescriptions, shipment information for one or more members of a group, and/or acquisition information for one or more prescription medicines. The example prescription medicine distribution system also includes a plurality of containers and a bulk supply of at least one prescription medicine.

Additional understanding of the inventive methods can be obtained by reviewing the detailed description of selected examples, below, with reference to the appended drawings.

### DESCRIPTION OF FIGURES

FIG. 1 is a flowchart representation of an example method for supplying prescription medicines to members of a group in a synchronized manner.

FIG. 2 is a flowchart representation of another example method for supplying prescription medicines to members of a group in a synchronized manner.

FIG. 3 is a flowchart representation of another example method for supplying prescription medicines to members of a group in a synchronized manner.

FIG. 4 is a schematic illustration of an example prescription medicine distribution system.

### DETAILED DESCRIPTION OF SELECTED EXAMPLES

The following detailed description and the appended drawings describe and illustrate various example methods for supplying prescription medicines to members of a group in a synchronized manner. The description and drawings are provided to enable one skilled in the art to practice example methods for supplying prescription medicines to members of a group in a synchronized manner. They are not intended to limit the scope of the claims in any manner.

As used herein, the term “day supply,” and grammatically related terms, refers to the number of units of a prescription medicine, such as pills, capsules, or another individual unit of a prescription medicine, necessary for proper dosing according to a prescription to a particular person for the prescription medicine over a twenty four (24) hour period.

As used herein, the term “delivery,” and grammatically related terms, refers to the act of leaving an object, such as a container, a parcel containing a container or containers, or other object, at a physical location for retrieval by a designated recipient.

As used herein, the term “fixed delivery date,” and grammatically related terms, refers to a date on which an object delivered to a designated recipient is expected to arrive at a designated physical location for retrieval by the designated recipient.

As used herein, the term “group,” and grammatically related terms, refers to a number of people sharing a common trait or a common relationship to each other, to another person, or to a legal entity, such as a business organization. For example, employees of a common employer constitute a group based on the common relationship each employee has to the employer.

As used herein, the term “next occurring fixed delivery date,” and grammatically related terms, refers to the fixed delivery date of one or more fixed delivery dates that will occur due to passage of time from a particular point in time, such as a particular date. For example, with a given date of January 1 and a list of fixed delivery dates that includes January 15, April 15, July 15, and October 15, the next occurring fixed delivery date from the given date is January 15.

As used herein, the term “prescription medicine,” and grammatically related terms, refers to a product intended to have a health benefit that requires a consumer to have a medical prescription to lawfully receive the product. The term includes pharmaceutical compositions, compounded preparations, medical devices, and any other product intended to have a health benefit that requires a medical prescription for a consumer to lawfully receive the product.

As used herein, the term “regular prescription supply,” and grammatically related terms, refers to a quantity of a prescription medicine that is necessary for proper dosing according to a prescription to a particular person for the prescription medicine over a determined regular prescription supply number of days.

As used herein, the term “sending,” and grammatically related terms, refers to the act of transferring an object, such as a container, a parcel containing a container or containers, or other object, for delivery to a designated recipient at a designated physical location.

As used herein, the term “supply interval,” and grammatically related terms, refers to a number of days that can be used to determine quantities of prescription medicines to provide people having prescriptions for the prescription medicines.

As used herein, the term “synchronized manner,” and grammatically related terms, refers to an act, result, or effect that occurs on the same date for members of a group. The term does not require that the act, result, or effect occur at the same time for members of the group.

As used herein, the term “year,” and grammatically related terms, relates to a period of twelve months. The term includes calendar years, which run from January 1 to December 31, and any other twelve month period of time.

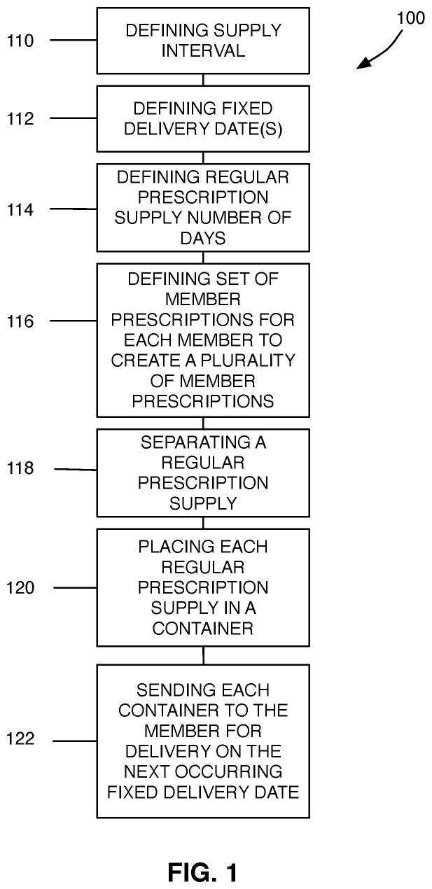

FIG. 1 illustrates a flowchart representation of an example method 100 for supplying prescription medicines to members of a group in a synchronized manner. The method 100 includes defining 110 a supply interval, defining 112 one or more fixed delivery dates for a given year based on the supply interval, defining 114 a regular prescription supply number of days, defining 116 a set of member prescriptions for each member of the group to create a plurality of member prescriptions, separating 118 a regular prescription supply of the relevant prescription medicine, which is a quantity of the prescribed medicine for the regular prescription supply number of days, from a bulk supply of the prescribed medicine for each member prescription of the plurality of member prescriptions to create a plurality of regular prescription supplies, placing 120 each regular prescription supply of the plurality of regular prescription supplies into a container, and sending 122 each container to the member of the group having the prescription for the regular prescription supply contained within the container for delivery on the next occurring fixed delivery date of the one or more fixed delivery dates.

Any suitable supply interval can be used in the defining 110 a supply interval. When performing this step, a skilled artisan will be able to define a supply interval suitable for use in a method according to a particular embodiment of the invention based on various considerations, including the availability of necessary quantities of relevant prescription medicines, desired shipping frequencies, and others. Examples of suitable supply intervals include 365 days, 180 days, 90 days, 30 days, 8 days, 7 days, 5days, 4 days, 3 days, 2 days, and 1 day. The inventor has determined that supply intervals of 90 days, 30 days, and 7 days are particularly advantageous as each represents a desirable balance of various considerations, including operational efficiencies and customer expectations. A supply interval of 90 days is considered particularly advantageous at least because it enables quarterly deliveries to member and charges to payers.

The fixed delivery dates defined in the step of defining 112 one or more fixed delivery dates for a given year are based on the supply interval defined in the step of defining 110 a supply interval. The delivery dates are represented by a month and day number, such as January 15. In this step, it is critical that the fixed delivery dates are defined such that the number of days between each sequential pair of fixed delivery dates is equal to or greater than the supply interval defined in step 110. Advantageously, the fixed delivery dates are defined such that the number of days between each sequential pair of fixed delivery dates is greater than the supply interval defined in step 110. It is considered particularly advantageous to define fixed delivery dates that minimize the variability in the number of days between pairs of sequential fixed delivery dates as doing so minimizes excess prescription medicine that is included in shipments to members of the group. It is also considered advantageous to define fixed delivery dates that have common day numbers in the respective months, which the inventor has determined helps customers remember when their respective prescription medicines will arrive. Based on these considerations, a skilled artisan will be able to define fixed delivery dates for use in a method according to a particular embodiment of the invention. As an example, the inventor has determined that, based on these considerations and a supply interval of 90 days, fixed delivery dates of January 15, April 15, July 15, and October 15 for any given year are suitable, and indeed are presently preferred, for use in a method according to an embodiment of the invention.

In this example method, defining 114 a regular prescription supply number of days is performed by calculating the actual number of days between each pair of sequential fixed delivery dates defined in step 112. In this example method, a regular prescription supply number of days is then defined as the number of days between sequential fixed delivery dates having the greatest value. For example, with fixed delivery dates of January 15, April 15, July 15, and October 15, the number of days between the first and second fixed delivery dates, in a non-leap year, is 91, the number of days between the second and third fixed delivery dates is 91, the number of days between the third and fourth fixed delivery dates is 92 days, and the number of days between the fourth fixed delivery date and the first fixed delivery date of the next sequential period of time is 92 days. Accordingly, in this example method, the regular prescription supply number of days would be defined as 92. Alternatively, a regular prescription supply number of days can be defined by dividing the total number of days in the relevant period of time by the total number of fixed delivery dates and, if the result is not a whole number, rounding the result to the next greatest whole number. For example, with the four fixed delivery dates mentioned above for use over a year, a regular prescription supply number of days is defined as 365/4=91.25. Rounding this to the next whole number provides a regular prescription supply number of days of 92.

Defining 116 a set of member prescriptions for each member of the group to create a plurality of member prescriptions is performed by reviewing prescription information for each member of the group to identify prescriptions that can be included in performance of the method. Each member of the group may have zero prescriptions, one prescription, or more than one prescription. All or some of the prescriptions for each member can be included in the set of member prescriptions for a particular member based on various considerations, including known and/or expected availability of the relevant prescription medicine, known or expected price fluctuation of the relevant prescription medicine, and other considerations. The sets of member prescriptions defined in this group create a plurality of member prescriptions for the entire group.

At this point in the method, member prescriptions are advantageously processed individually. For example, for each member prescription of the plurality of member prescriptions, separating step 118 is performed to separate a regular prescription supply of the relevant prescription medicine from a bulk supply of the prescribed medicine. The regular prescription supply is a quantity of the relevant prescription medicine that is necessary for proper dosing of the relevant member, based on the dosage instructions in the relevant prescription, over the regular prescription supply number of days. For example, if the regular prescription supply number of days defined in step 114 is 92 days, separating step 118 is performed for each member prescription such that a quantity of the relevant prescription medicine corresponding to a 92 day supply is separated from a bulk supply of the prescription medicine. The actual quantity of the units, such as pills, capsules, or other relevant unit, of the prescription medicine to be included in the regular prescription supply can be determined by multiplying the day supply for the prescription medicine by the regular prescription supply number of days defined in step 114. The physical act of separating a quantity from a bulk supply can be performed using any suitable techniques, tools, and/or equipment, including, for example, spatulas, sorters, scales, automated techniques, and manual techniques.

Placing 120 each regular prescription supply of the plurality of regular prescription supplies into a container is performed for each member prescription by placing the relevant regular prescription supply, produced in step 118, into a suitable container. Any container suitable for containing the relevant prescription medicine can be used and a skilled artisan will be able to select an appropriate container for each regular prescription supply based on various considerations, including the nature of the prescription medicine, expected conditions during shipment, any handling and/or storage requirements for the prescription medicine, and other considerations. Examples of suitable containers include, but are not limited to, blister packs, vials, envelopes, dropper-cap vials, multi-chamber containers, and bottles. In some embodiments, it can be advantageous to combine regular prescription supplies for multiple prescriptions for the same member of the group into a single container. For example, multiple regular prescription supplies for a member can be placed into a blister pack with individual doses of the prescription medicines chambered together, as appropriate, and organized by dose day and time.

Sending 122 each container to the member of the group having the prescription for the regular prescription supply contained within the container is performed by transmitting the container to a physical address on record for the member such that the container is scheduled to be delivered to the address on the next occurring fixed delivery date of the one or more fixed delivery dates. In this step, sending can be accomplished using any suitable technique for sending the relevant prescription medicine(s) to the member, such as in-person delivery, delivery by courier, such as a delivery service like FedEx, UPS, DHL, or the like, delivery through the United States Postal Service, delivery by an autonomous robotic device, and delivery by a remotely operated drone. Also in this step, multiple containers that contain regular prescription supplies for the same member can be combined and sent to the member in one step, such as in one master container that contains the multiple containers.

Additional steps can be included when performing the method 100. For example, a step of charging a participation fee to a common prescription payer for the group can be included. This step is advantageously included when a common prescription payer has an obligation to pay for prescribed medicines for all members of the group, such as an employer for which all members are employees. If included, the participation fee is advantageously based on the number of members of the group or the number of members of the group having member prescriptions that will be included in performance of the method. The participation fee can be a one time fee or a recurring fee, such as a quarterly fee.

The method can also include charging a prescription fee for each member prescription of the plurality of member prescriptions to a common prescription payer for the group. In examples that include charging a participation fee to the common prescription payer for the group, the prescription fee advantageously reflects the cost paid by the performer of the method to a manufacturer of the prescription medicine or an upstream supplier of the prescription medicine. In this sense, the charging of a participation fee enables the performer of the method to provide prescription medicines at cost.

While the inclusion of one or both of the optional steps of charging a participation fee and charging a prescription fee is advantageous in methods in which a common payer is involved, one or both of these steps can be included in methods according to embodiments in which individual members are responsible for payment of fees. For example, each of the optional steps, whether one or both are included in the method, of charging a participation fee and charging a prescription fee can be performed in a manner that charges the individual fee(s) to individual members of the group. Indeed, this can be advantageous for methods be performed for certain groups, such as groups of individuals that do not have access to medical insurance or medical insurance that includes a prescription medicine benefit.

In methods in which the one or more fixed delivery dates comprises more than one fixed delivery dates, the separating 118, placing 120, and sending 122 are advantageously repeated for each remaining fixed delivery date of the one or more fixed delivery dates over the course of a defined period, such as a year. The separating and placing for each repeating cycle can be done at the same time as the initial separating and placing, or later. It is critical, however, that the step 120 of sending each container to the member of the group always be performed such that each container be delivered to the member on the next occurring fixed delivery date of the one or more fixed delivery dates.

Performance of the method 100 may be initiated for a group at any time during a given period. For example, in some examples, the method is initiated just prior to one of the fixed delivery dates such that the initial delivery to each member of the group includes a regular prescription supply for each member prescription. In some examples, however, the time between a planned initiation of performance of the method and the next occurring fixed delivery date is considered too long to wait, for convenience, medical reasons, or otherwise. In these examples, an initial delivery to each member of the group can include an initial prescription supply for each of the member prescription(s) for the relevant member. The initial prescription supply is a quantity of the relevant prescription medicine that is necessary for proper dosing of the relevant member, based on the dosage instructions in the relevant member prescription, over the number of days between an initial delivery date for the prescription medicine and the next occurring fixed delivery date. The use of an initial prescription supply can be useful when first initiating performance of the method 100 for a group or when adding a member having one or more member prescriptions to a group for which an ongoing performance of the method 100 is in process. For example, if a member having one or more member prescriptions is added to a group for which the method 100 is already being performed, an initial delivery to the new member can include an initial prescription supply for each member prescription for the new member, the initial prescription supply being a quantity of the relevant prescription medicine that is necessary for proper dosing of the new member, based on the dosage instructions in the relevant prescription, over the number of days between an initial delivery date for the prescription medicine and the next occurring fixed delivery date. As with the regular prescription supply, the actual quantity of the units, such as pills, capsules, or other relevant unit, of the prescription medicine to be included in the initial prescription supply can be determined by multiplying the day supply for the prescription medicine by the number of days between an initial delivery date for the prescription medicine and the next occurring fixed delivery date.

Medication regimes can change, of course, and such changes, frequently, need to be implemented immediately and cannot wait until the next fixed delivery date. If a dosing regimen for a particular member prescription is changed such that the member needs to take a lower daily dose of the relevant prescription medicine, the regular prescription supply can be updated accordingly for the member for the next occurring fixed delivery date. If, however, a dosing regimen for a particular member prescription is changed such that the relevant member needs to take a higher daily dose of the relevant prescription medicine, the quantity of the prescription medicine that the user has on hand may be insufficient to last until the next occurring fixed delivery date. In this circumstance, an interim delivery including a supplemental prescription supply of the relevant prescription medicine can be sent to the member for the relevant member prescription. The supplemental prescription supply is, at a minimum, a quantity of the relevant prescription medicine that, when combined with the quantity of the relevant prescription medicine that the member has on hand, is necessary for proper dosing of the relevant member, based on the modified dosage regimen, over the number of days between a delivery date for the supplemental prescription supply and the next occurring fixed delivery date. At a maximum, the supplemental prescription supply is a quantity of the relevant prescription medicine that is necessary for proper dosing of the relevant member, based on the modified dosage regimen, over the number of days between a delivery date for the supplemental prescription supply and the next occurring fixed delivery date.

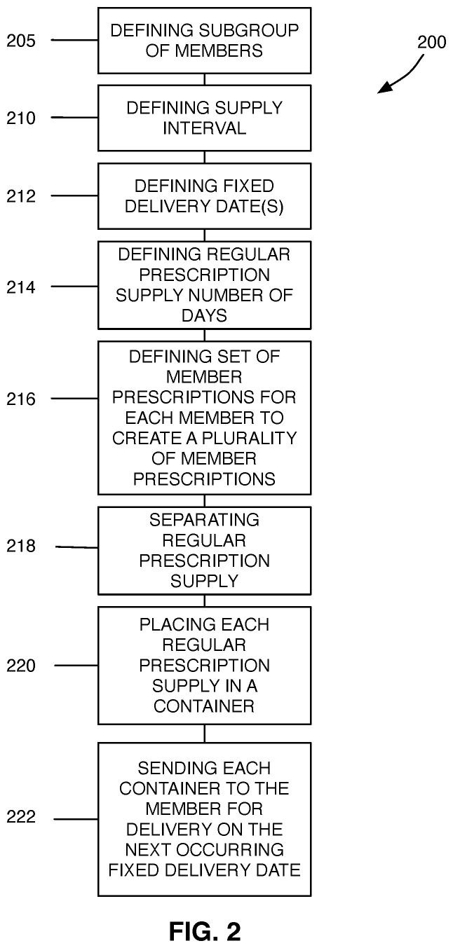

FIG. 2 illustrates a flowchart representation of another example method 200 for supplying prescription medicines to members of a group in a synchronized manner. The method 200 is similar to the method 100 describe above and illustrated in FIG. 1. Accordingly, the method 200 includes defining 210 a supply interval, defining 212 one or more fixed delivery dates for a given year based on the supply interval, defining 214 a regular prescription supply number of days, defining 216 a set of member prescriptions for each member in a defined subgroup of members (see below) to create a plurality of member prescriptions, separating 218 a regular prescription supply of the relevant prescription medicine, which is a quantity of the prescribed medicine for the regular prescription supply number of days, from a bulk supply of the prescribed medicine for each member prescription of the plurality of member prescriptions to create a plurality of regular prescription supplies, placing 220 each regular prescription supply of the plurality of regular prescription supplies into a container, and sending 222 each container to the member having the prescription for the regular prescription supply contained within the container for delivery on the next occurring fixed delivery date of the one or more fixed delivery dates.

In this example, a step 205 of defining a subgroup of members from all members of the group for participation in the method is included. This step 205 is advantageously performed prior to defining 216 a set of member prescriptions to create a plurality of member prescriptions, which, in this example, is performed only for each member of the defined subgroup. Inclusion of this step 205 can be advantageous when it is desired to limit performance of the method to include only those members of the group having member prescriptions for certain types of prescription medicines, such as those having relatively high cost or having relatively high price variability. Inclusion of this step can also be advantageous when it is desired to limit performance of the method to include only those members of the group having multiple prescriptions or having more than a threshold number of prescriptions, such as three, four, five, or more than five prescriptions.

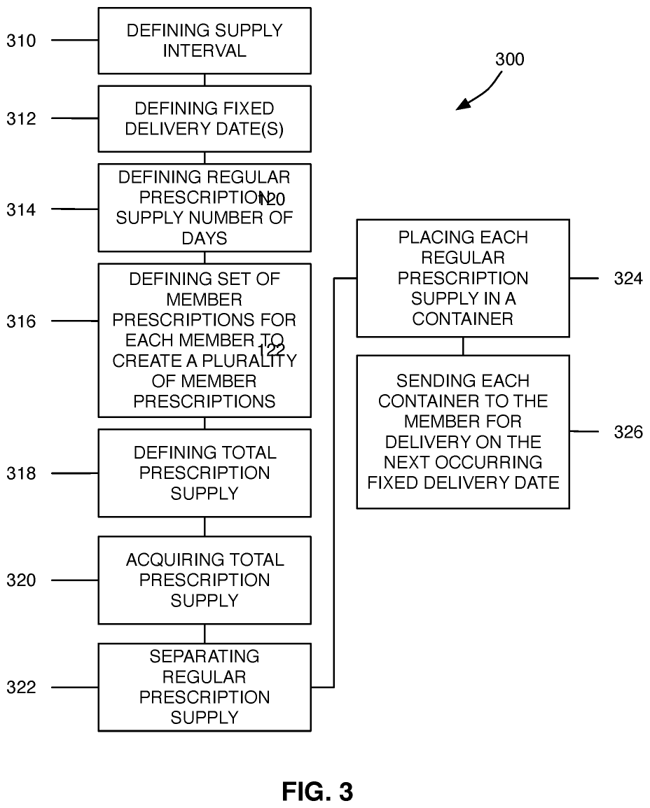

FIG. 3 illustrates a flowchart representation of another example method 300 for supplying prescription medicines to members of a group in a synchronized manner. The method 300 includes defining 310 a supply interval, defining 312 one or more fixed delivery dates for a given year based on the supply interval, defining 314 a regular prescription supply number of days, defining 316 a set of member prescriptions for each member of the group to create a plurality of member prescriptions, for each prescription medicine represented in the plurality of member prescriptions, defining 318 a total prescription supply, for each prescription medicine represented in the plurality of member prescriptions, acquiring 320 the total prescription supply from a supplier of the prescription medicine, such as a manufacturer or a distributor, separating 322 a regular prescription supply of the relevant prescription medicine, which is a quantity of the prescribed medicine for the regular prescription supply number of days, from the total prescription supply of the prescribed medicine for each member prescription of the plurality of member prescriptions to create a plurality of regular prescription supplies, placing 324 each regular prescription supply of the plurality of regular prescription supplies into a container, and sending 326 each container to the member of the group having the prescription for the regular prescription supply contained within the container for delivery on the next occurring fixed delivery date of the one or more fixed delivery dates.

In this example, the method 300 includes the steps of defining 318 a total prescription supply and acquiring 320 the total prescription supply for each prescription medicine represented in the plurality of member prescriptions. The total prescription supply is, at a minimum, a quantity of the relevant prescription medicine necessary to provide required regular prescription supply(ies), initial prescription supply(ies), and/or supplemental prescription supply(ies) for the next occurring fixed delivery date. Steps 318 and 320 can be performed at any suitable time during performance of the method. As noted above in connection with example method 100, when the one or more fixed delivery dates comprises more than one fixed delivery date, the separating 322, placing 324, and sending 326 are advantageously repeated for each remaining fixed delivery date of the one or more fixed delivery dates over the course of a defined period, such as a year. In these methods, it is considered advantageous, that step 318 be performed only once between each repetition of the separating 322, placing 324, and sending 326 steps. Furthermore, it is considered particularly advantageous that the step 320 also be performed only once between each repetition of the separating 322, placing 324, and sending 326 steps. Performing the method 300 in this manner allows a performer of the method 300 to minimize resources dedicated to storing and handling inventory of prescription medicines between fixed delivery dates.

In one example method, the step of acquiring 320 is performed such that at least one prescription medicine of all prescription medicines represented in the plurality of member prescriptions is acquired when the quantity of the prescription medicine being acquired has between 100% of the regular prescription supply number of days and 125% of the regular prescription supply number of days remaining prior to its expiration date. Performing this step in this manner assures that regular prescription supplies of the prescription medicine can be delivered to the relevant members with a sufficient portion of the life cycle of the prescription medicine remaining before its expiration while also allowing a performed of the method 300 to seek end of life cycle discounts on pricing for the relevant prescription medicine as part of the acquiring 320 step. It is also considered advantageous to perform the acquiring step 320 such that at least one prescription medicine of all prescription medicines represented in the plurality of member prescriptions is acquired when the quantity of the prescription medicine being acquired has between 100% of the regular prescription supply number of days and 120% of the regular prescription supply number of days remaining prior to its expiration date. It is also considered advantageous to perform the acquiring step 320 such that at least one prescription medicine of all prescription medicines represented in the plurality of member prescriptions is acquired when the quantity of the prescription medicine being acquired has between 100% of the regular prescription supply number of days and 115% of the regular prescription supply number of days remaining prior to its expiration date. It is also considered advantageous to perform the acquiring step 320 such that at least one prescription medicine of all prescription medicines represented in the plurality of member prescriptions is acquired when the quantity of the prescription medicine being acquired has between 100% of the regular prescription supply number of days and 110% of the regular prescription supply number of days remaining prior to its expiration date. It is also considered advantageous to perform the acquiring step 320 such that at least one prescription medicine of all prescription medicines represented in the plurality of member prescriptions is acquired when the quantity of the prescription medicine being acquired has between 100% of the regular prescription supply number of days and 105% of the regular prescription supply number of days remaining prior to its expiration date. It is also considered advantageous to perform the acquiring step 320 such that at least one prescription medicine of all prescription medicines represented in the plurality of member prescriptions is acquired when the quantity of the prescription medicine being acquired has about 110% of the regular prescription supply number of days remaining prior to its expiration date. It is also considered advantageous to perform the acquiring step 320 such that at least one prescription medicine of all prescription medicines represented in the plurality of member prescriptions is acquired when the quantity of the prescription medicine being acquired has about 105% of the regular prescription supply number of days remaining prior to its expiration date. It is also considered advantageous to perform the acquiring step 320 such that at least one prescription medicine of all prescription medicines represented in the plurality of member prescriptions is acquired when the quantity of the prescription medicine being acquired has about 101% of the regular prescription supply number of days remaining prior to its expiration date.

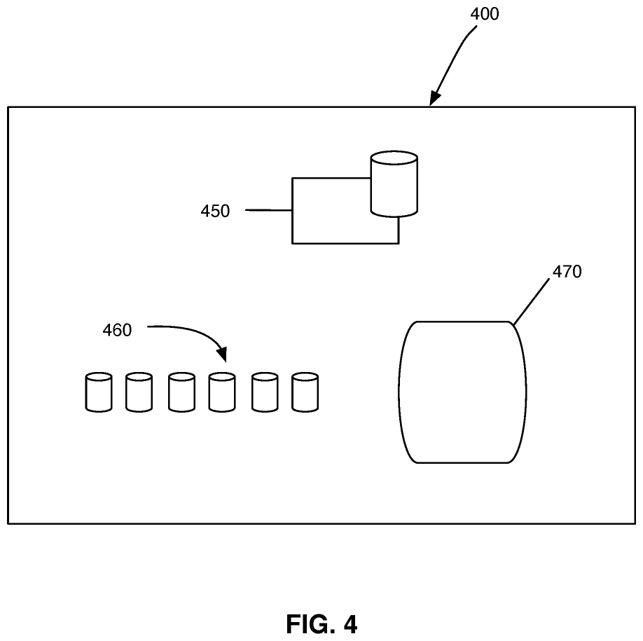

FIG. 4 illustrates an example prescription medicine distribution system 400. The system includes a computer 450 having a processor, a computer readable memory, a database server and/or client application adapted to access a database server application, and computer program instructions that, when executed by the processor, retrieve data from the database server application, either directly or via the database client application, relating to a supply interval, one or more fixed delivery dates, one or more member prescriptions, shipment information for one or more members of a group, and/or acquisition information for one or more prescription medicines, as described herein. The system 400 also includes a plurality of containers 460, each suitable for containing at least a regular prescription supply, an initial prescription supply, and/or a supplemental prescription supply as described herein. The system 400 also includes a bulk supply 470 of at least one prescription medicine. A user can use the system 400 in performance of a method according to an embodiment, such as one or more of the example methods described herein.

The computer can be a general purpose computer, special purpose computer, and/or other programmable computing machine. The computer program instructions can be stored on a computer-usable or computer-readable storage medium, such as an electronic, magnetic, optical, electromagnetic, infrared, or semiconductor system, apparatus, device, or propagation medium, including, but not limited to, a portable computer disk, a portable computer drive, a portable computer storage device, a random access memory (RAM), a read-only memory (ROM), an erasable programmable read-only memory (EPROM or Flash memory), an optical fiber, and a portable compact disc read-only memory (CD-ROM). The database server and/or client application can be any computer application suitable for storing member prescription information in a manner that permits retrieval of the member prescription information during performance of a method according to an embodiment, such as the example methods described herein.

The plurality of containers 460 includes a quantity of containers, at a minimum, that is sufficient for a user of the system to place a regular prescription supply, an initial prescription supply, or a supplemental prescription supply, as described herein, for each member prescription for a fixed delivery date into an individual container.

The bulk supply 470 of at least one prescription medicine is a quantity, at a minimum, of the at least one prescription medicine that is necessary to provide required regular prescription supply(ies), initial prescription supply(ies), and/or supplemental prescription supply(ies) for the next occurring fixed delivery date. The bulk supply 470 can be a total prescription supply as described above in connection with example method 300.

The foregoing detailed description provides examples of the invention and includes the best mode for practicing the invention. The description and illustration of the example methods are intended only to provide examples of the invention and not to limit the scope of the invention, or its protection, in any manner.

I claim:

1. A method for supplying prescription medicines to members of a group in a synchronized manner, said method comprising:
defining a supply interval;
defining one or more fixed delivery dates for a given year based on the supply interval;
defining a regular prescription supply number of days;
defining a set of member prescriptions for each member of said group to create a plurality of member prescriptions, each member prescription of the plurality of member prescriptions including dosing instructions for a prescription medicine and the member;
for each prescription medicine represented in the plurality of member prescriptions, defining a total prescription supply;
for each prescription medicine represented in the plurality of member prescriptions, acquiring the total prescription supply from a supplier of the prescription medicine;
for each member prescription of the plurality of member prescriptions, separating a regular prescription supply of the relevant prescription medicine from the total prescription supply of the prescription medicine to create a plurality of regular prescription supplies;
placing each regular prescription supply of the plurality of regular prescription supplies into a container; and
sending each container to the member of the group having the prescription for the regular prescription supply contained within the container for delivery on the next occurring fixed delivery date of the one or more fixed delivery dates so that all regular prescription supplies of the plurality of regular prescription supplies are scheduled for delivery to a corresponding member of the group on the next occurring fixed delivery date of the one or more fixed delivery dates.

2. The method of claim 1, wherein a common prescription payer has an obligation to pay for prescribed medicines for all members of said group.

3. The method of claim 2, wherein the common prescription payer is an employer and each member of said group is an employee the employer.

4. The method of claim 2, further comprising charging a participation fee to the common prescription payer.

5. The method of claim 4, wherein the participation fee is based on the number of members of said group.

6. The method of claim 5, wherein the step of charging a participation fee is performed only once as a one time fee.

7. The method of claim 5, wherein the step of charging a participation fee is performed more than once.

8. The method of claim 7, wherein the step of charging a participation fee is performed once prior to each fixed delivery date of the one or more fixed delivery dates.

9. The method of claim 2, further comprising charging a prescription fee for each member prescription of the plurality of member prescriptions to the common prescription payer.

10. The method of claim 1, wherein each set of member prescriptions comprises more than one member prescription.

11. The method of claim 1, wherein each set of member prescriptions comprises three, four, five, or more than five member prescriptions.

12. The method of claim 1, wherein the regular prescription supply number of days is the number of days between sequential fixed delivery dates of the one or more fixed delivery dates having the greatest value.

13. A method for supplying prescription medicines to members of a group in a synchronized manner, said method comprising:
defining a supply interval;
defining one or more fixed delivery dates for a given year based on the supply interval;
defining a regular prescription supply number of days;
defining a set of member prescriptions for each member of said group to create a plurality of member prescriptions, each member prescription of the plurality of member prescriptions including dosing instructions for a prescription medicine and the member;
for each prescription medicine represented in the plurality of member prescriptions, defining a total prescription supply;
for each prescription medicine represented in the plurality of member prescriptions, acquiring the total prescription supply from a supplier of the prescription medicine such that the acquiring is completed on a date when the quantity of the prescription medicine being acquired has between 100% of the regular prescription supply number of days and 125% of the regular prescription supply number of days remaining prior to an expiration date of the quantity of the prescription medicine being acquired;
for each member prescription of the plurality of member prescriptions, separating a regular prescription supply of the relevant prescription medicine from the total prescription supply of the prescription medicine to create a plurality of regular prescription supplies;
placing each regular prescription supply of the plurality of regular prescription supplies into a container; and
sending each container to the member of the group having the prescription for the regular prescription supply contained within the container for delivery on the next occurring fixed delivery date of the one or more fixed delivery dates so that all regular prescription supplies of the plurality of regular prescription supplies are scheduled for delivery to a corresponding member of the group on the next occurring fixed delivery date of the one or more fixed delivery dates.

14. The method of claim 13, wherein the step of acquiring the total prescription supply from a supplier of the prescription medicine is completed on a date when the quantity of the prescription medicine being acquired has between 100% of the regular prescription supply number of days and 120% of the regular prescription supply number of days remaining prior to an expiration date of the quantity of the prescription medicine being acquired.

15. The method of claim 13, wherein the step of acquiring the total prescription supply from a supplier of the prescription medicine is completed on a date when the quantity of the prescription medicine being acquired has between 100% of the regular prescription supply number of days and 115% of the regular prescription supply number of days remaining prior to an expiration date of the quantity of the prescription medicine being acquired.

16. The method of claim 13, wherein the step of acquiring the total prescription supply from a supplier of the prescription medicine is completed on a date when the quantity of the prescription medicine being acquired has between 100% of the regular prescription supply number of days and 110% of the regular prescription supply number of days remaining prior to an expiration date of the quantity of the prescription medicine being acquired.

17. The method of claim 13, wherein the step of acquiring the total prescription supply from a supplier of the prescription medicine is completed on a date when the quantity of the prescription medicine being acquired has between 100% of the regular prescription supply number of days and 105% of the regular prescription supply number of days remaining prior to an expiration date of the quantity of the prescription medicine being acquired.

18. A method for supplying prescription medicines to members of a group in a synchronized manner, said method comprising:
defining a subgroup of members from all members of said group;
defining a supply interval;
defining one or more fixed delivery dates for a given year based on the supply interval;
defining a regular prescription supply number of days;
defining a set of member prescriptions for each member in the subgroup of members of said group to create a plurality of member prescriptions, each set of member prescriptions comprising more than one member prescription;
separating a regular prescription supply of the relevant prescription medicine from a bulk supply of the prescribed medicine for each member prescription of the plurality of member prescriptions to create a plurality of regular prescription supplies;
placing each regular prescription supply of the plurality of regular prescription supplies into a container; and
sending each container to the member of the group having the prescription for the regular prescription supply contained within the container for delivery on the next occurring fixed delivery date of the one or more fixed delivery dates.

19. The method of claim 18, wherein each set of member prescriptions comprises three, four, five, or more than five member prescriptions.

20. The method of claim 18, further comprising:
for each prescription medicine represented in the plurality of member prescriptions, defining a total prescription supply;
for each prescription medicine represented in the plurality of member prescriptions, acquiring the total prescription supply from a supplier of the prescription medicine such that the acquiring is completed on a date when the quantity of the prescription medicine being acquired has between 100% of the regular prescription supply number of days and 125% of the regular prescription supply number of days remaining prior to an expiration date of the quantity of the prescription medicine being acquired.
